(19) United States
(12) Patent Application Publication
(10) Pub. No.: US 2005/0089474 A1
(43) Pub. Date: Apr. 28, 2005

(54) Method and system for producing magnetic resonance images

(76) Inventors: Yannick Cremillieux, Lyon (FR); Emmanuelle Canet, Lyon (FR); Virginie Callot, Lyon (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/488,935

(22) PCT Filed: Apr. 25, 2002
(86) PCT No.: PCT/FR02/01430

(30) Foreign Application Priority Data
Apr. 25, 2001 (FR) 01/05555

(51) Int. Cl. A61K 49/00; A61B 5/055

(52) U.S. Cl. 424/9.3; 600/410

(57) ABSTRACT

The invention relates to a process for the production of magnetic resonance images of a body for which an image is required, characterized in that it comprises the following steps: place at least the body for which an image is required in a stationary magnetic field, inject a blood tracer containing at least one hyperpolarized rare gas into the blood circulatory system for at least the body for which an image is required, apply radio frequency pulses and magnetic field gradients to the body for which an image is required to excite nuclear magnetization of the hyperpolarized rare gas, acquire nuclear magnetic resonance signals in the k space in the form of N acquisitions each passing through the centre of the k space, and, starting from nuclear magnetic resonance signals, reconstruct images, replacing at least one acquisition of a k space for each image by an acquisition of the next k space.

The technical domain of this invention is magnetic resonance imagery of a body for which an image in the general sense, such as part of a human being and particularly the heart, is required.

More precisely, the technical domain of the invention is magnetic resonance imagery using rare gases, called hyperpolarized gases.

Conventionally, the production of nuclear magnetic resonance images (NMR) requires the acquisition of NMR signals from the body for which an image is required. An appropriate application of radio frequency pulses and magnetic field gradients during these acquisitions, provides a means of localizing the source of the NMR signal.

The procedure for obtaining a two-dimensional image of the body for which an image is required consists of making an imagery sequence during which a sequential combination of magnetic field gradients and radio frequency pulses is made. This imagery sequence is applied such that all sampled values of the NMR signal fill the plane, called the Fourier plane or the k space, in the MRI terminology. A Fourier transform operation is then carried out on the sampled NMR signals to produce an image of the distribution of nuclear magnetizations in the body for which an image is required.

The Fourier plane is produced by taking a large number of imagery sequences. The combination used in most cases is so-called Fourier imagery with a Cartesian distribution of points sampled sequentially along parallel lines in the Fourier plane. Each new image is formed from complete acquisition of a new Fourier plane. Therefore the time resolution of an imagery sequence corresponds to the acquisition time of the Fourier plane, in other words the product N.TR where N is the number of lines in the Fourier space and TR is the repetition time separating acquisition of two lines.

An NMR fluoroscopy technique is proposed which in theory can considerably improve the resolution of the imagery sequence in time (RIEDERER et al., Magnetic Resonance Medicine, 8-15, 1988). The principle consists of reconstructing NMR images using signals belonging to different Fourier spaces. Thus, for example an image can be made using the last N−1 lines of a k space and the first line of the next k space. The process can be repeated by further offsetting the beginning of the k space used by one line, such that the result is that the last N−2 lines of the first k space, and the first two lines of the second k space are retained.

The resolution in time is improved, in the sense in which each reconstructed image is therefore offset in time from the first by a time TR. This method is known in the MRI field as a “sliding window”. In practice, this method has many disadvantages. The general shape of the image is given essentially by central lines of the k space containing low spatial frequencies of the body for which an image is required (external lines of the k space correspond to spatial high frequencies giving details and the resolution in space of the body for which an image is required). Consequently, any variation of the shape, intensity and position of the body for which an image is required that takes place during acquisitions of peripheral lines, is not recorded. Thus, large discontinuities occur generating artifacts in the series of dynamic images.

In order to use the sliding window technique much more efficiently, known art includes a method of calling upon other imagery techniques such as imagery sequences, called spiral and projection/reconstruction techniques. These imagery sequences have in common the fact that the centre of the k space is acquired for each repetition time TR. Signals are acquired along radial lines in the projection/reconstruction technique and along spiral lines in the spiral imagery technique. The passage through the centre of the k space in each repetition time TR prevents discontinuities in monitoring dynamic changes to the body for which an image is required. The sliding window technique has been proposed and applied for spiral imagery, as described particularly in U.S. Pat. No. 5,485,086 and in projection/reconstruction, as described particularly in U.S. Pat. No. 5,502,385.

Although these imagery techniques can further improve the resolution of images, in practice they are not suitable for obtaining images, particularly of the heart, and also require synchronized acquisition of NMR signals on the heart cycle. Thus, these techniques cannot give a correct imagery of coronaries and infusion of the myocardium.

Obviously, the state of the art includes the use of digital angiography (X-rays) for imagery of coronaries and scintigraphy with thallium (gamma camera) for infusion imagery. However, these two complementary examinations require different imagery methods each using ionising radiation.

Therefore, there is a need for an imagery technique, particularly imagery of the heart and particularly coronaries and infusion of the myocardium, which can be used independently and can overcome problems related to the use of ionising radiation, while obtaining excellent quality images.

In order to satisfy this need, the purpose of the invention relates to a process for the production of magnetic resonance images of a body for which an image is required, characterized in that it comprises the following steps:
place at least the body for which an image is required in a stationary magnetic field,
inject a blood tracer containing at least one hyperpolarized rare gas into the blood circulatory system for at least the body for which an image is required,
apply radio frequency pulses and magnetic field gradients to the body for which an image is required to excite nuclear magnetization of the hyperpolarized rare gas and thus obtain emission of nuclear magnetic resonance signals,
acquire nuclear magnetic resonance signals in the k space in the form of N acquisitions each passing through the centre of the k space and with an acquisition time N.TR for each k space (where TR is the repetition time separating two acquisitions),
and, starting from nuclear magnetic resonance signals, reconstruct images, replacing at least one acquisition of a k space for each image by an acquisition of the next k space.

Therefore, the purpose of the invention is to combine use of the so-called sliding window method in projection/reconstruction or in spiral imagery, with the use of blood tracers based on hyperpolarized rare gases.

Another purpose of the invention is to propose an installation for the production of magnetic resonance images of a body for which an image is required, characterized in that it comprises:
means of injecting a blood tracer containing at least one hyperpolarized rare gas into the blood circulatory system of at least one body for which an image is required,
and a device for the production of magnetic resonance images comprising:
   means of production of a stationary magnetic field,
   a high frequency coil system in order to produce radio frequency pulses,
   a gradient coil system in order to produce magnetic fields with gradients,
   means of acquisition of nuclear magnetic resonance signals in the k space in the form of N acquisitions each passing through the centre of the k space and with an acquisition time N.TR for each k space (where TR is the repetition time separating two acquisitions),
   and means of reconstructing images starting from nuclear magnetic resonance signals, each replacing at least one acquisition of a k space by an acquisition of a next k space.

According to another characteristic of the invention, the magnetic resonance image production device comprises means of cyclically varying the normal direction to the imagery plane in which the magnetic field gradients used to acquire the k space are applied.

According to another characteristic of the invention, the magnetic resonance image production device comprises means of varying the normal direction to the imagery plane in which the magnetic field gradients used to acquire the k space are applied, for each N acquisition.

The image production installation according to the invention comprises firstly means of injecting a blood tracer according to the invention into the circulatory system, and also a device for the production of magnetic resonance images.

The injection means are all of a known type to enable a blood tracer to pass into the circulatory system of at least the body for which an image is required. According to the invention, the blood tracer contains at least a hyperpolarized rare gas such as helium 3 or xenon 129. These rare gases are said to be hyperpolarized because they are previously subjected to an optical pumping technique to preferably orient their nuclear magnetization in a given direction. According to this polarization process, the nuclear magnetic resonance (NMR) signals of these rare gases are multiplied by several orders of magnitude. These hyperpolarized rare gases are known to persons skilled in the art and are described particularly in the following articles: G. D. CATES et al., Phys. Rev. A 45 (1992), 4631, M. (1985), 260), L. D. SCHAERER, Phys. Lett. 180 (1969), 83: F. LALOE et al., AIP Conf. Proc. # 131 (Workshop on Polarized 3He Beams and Targets, 1984).

This type of rare gas is used in an emulsion or in a solution or after encapsulation in micro bubbles.

The installation according to the invention also comprises a device for the production of images by nuclear magnetic resonance, using the imagery technique known as the sliding window in projection/reconstruction technique, as described particularly in U.S. Pat. No. 5,502,385 or the spiral sliding window imagery technique as described particularly in U.S. Pat. No. 5,485,086. This type of image production device will be described briefly in the rest of this description since it is well known to a person skilled in the art.

Conventionally, this type of image production device comprises means of production of a homogenous stationary magnetic field composed of coils arranged concentrically around a preferred axis and arranged on a spherical surface inside which the patient to be examined is placed. Conventionally, this type of device also comprises a system of gradient coils for the production of magnetic fields with gradients moving in the three directions in space. Furthermore, this type of production system comprises a high frequency coil system for the production of radio frequency pulses.

Conventionally, the production device comprises control means of creating a sequential combination of magnetic field gradients and radio frequency pulses, such that the set of sampled values of the NMR signal fill the “Fourier” plane or the k space.

The production device also comprises acquisition means in the k space of nuclear magnetic resonance signals produced by the blood tracer. According to the invention, nuclear magnetic resonance signals are recorded in the form of N acquisitions each passing through the centre of the k space and with an acquisition time N.TR for each k space, where TR is the repetition time separating two acquisitions.

If the imagery technique (also called the projection/reconstruction technique) is used, the nuclear magnetic resonance signals are acquired in the form of N line acquisitions each passing through the centre of the k space. If the spiral imagery principle is used, the nuclear magnetic resonance signals are acquired in the form of N acquisitions of spiral curves each passing through the centre of the k space.

The production device also comprises image reconstruction means, starting from nuclear magnetic resonance signals, adapted to use the so-called sliding window technique, for which the reconstruction principle is to use signals belonging to different Fourier spaces. Thus, for each image, at least one acquisition of a k space is replaced by an acquisition of a next k space. According to one preferred characteristic of the invention, this sliding window technique is designed to build up images by replacing at least the first acquisition of a k space for each image by the first acquisition of a next k space. Thus, as described above, an image can be reconstructed using the last N−1 acquisitions of a k space and the first acquisition of the next k space. This process may be repeated by once again offsetting the start of the k space used by one acquisition and thus retaining the last N−2 acquisitions of the first k space and the first two acquisitions of the second k space.

The device described above enables the use of a method of producing magnetic resonance images of a body for which an image is required. Acquisition of NMR signals in projection/reconstruction or spiral is triggered before or during the passage of the blood tracer in the organ for which the image is required and is applied continuously throughout the time that the blood tracer is passing.

The advantage of a blood tracer based on hyperpolarized rare gases lies in the fact that the display of the blood vessels is not disturbed by the presence of the proton NMR signal from the surrounding environment (tissue, blood, etc.). Imagery of the distribution of a blood tracer based on hyperpolarized rare gases is based on the measurement of their own NMR signal (helium 3 or xenon 129 nucleus). In this case, the image obtained is a direct measurement of the intravascular distribution of rare gases. Thus, this method is similar to the concept of a radioactive tracer used in nuclear medicine (scintigraphy imagery, tomography by emission of positons, etc.), and is thus quite different from the method of using contrast agents used in the past in MRI and based on a measurement of their indirect effect on the NMR signal of protons in the surrounding environment.

To the extent that the display of blood vessels is not disturbed by the presence of the proton NMR signal in the surrounding environment, the sliding window technique can be used for imagery of coronary arteries without synchronization of the NMR acquisition on the cardiac cycle. Thus, images can be obtained in all section planes or in projection (without selecting the section) of coronary vessels and microcirculation of the myocardium.

Moreover, use of the sliding window technique for dynamic imagery of coronaries using rare gases is justified by the shape of variations of the signal with time. As the blood tracer passes through the blood vessels, variations of intensity in the image are in the form of a progressive filling of blood vessels by the blood tracer. This bolus passage is fast compared with the total acquisition time of the image and cannot be satisfactorily determined using conventional imagery techniques. On the other hand, the sliding window technique provides a means of reconstructing images separated only by a repetition time. Therefore, this technique provides a means of monitoring progression of the NMR signal for rare gases inside the vascular sector.

The particular properties of a blood tracer based on hyperpolarized rare gases provides means of obtaining series of images with a high resolution in time and space, particularly for the heart, but also for other regions of interest such as lungs, brain, kidneys, etc., using the sliding window technique.

According to one variant embodiment, the process according to the invention is designed to obtain different section planes of the body for which an image is required during the same injection. To achieve this, the sliding window technique is modified so as to obtain different orientations during the same injection of the blood tracer based on rare gas. Remember that the projection plane of the image is given by the plane containing all directions of imagery gradients used.

According to a first solution, gradient direction choices are interlaced so that NMR acquisitions obtained correspond for example alternately to coronal and transverse sections. Thus, it is planned to cyclically vary the normal direction to the imagery plane in which magnetic field gradients are applied, to acquire the k space. This alternation in the projection plane is made at the detriment of the resolution of the sliding window technique with time.

Another solution is to vary the normal direction to the imagery plane in which magnetic field gradients used to acquire the k space are applied, for each new N acquisition. The series of reconstructed dynamic images can thus progressively move for example from a coronal plane to a transverse plane. This solution does not deteriorate the resolution of the series of images with time, but the gradual reorientation of the orientation of the projection plane takes place at the detriment of the spatial resolution of images.

1. Process for the production of magnetic resonance images of a body for which an image is required, characterized in that it comprises the following steps:
place at least the body for which an image is required in a stationary magnetic field,
inject a blood tracer containing at least one hyperpolarized rare gas into the blood circulatory system for at least the body for which an image is required,
apply radio frequency pulses and magnetic field gradients to the body for which an image is required to excite nuclear magnetization of the hyperpolarized rare gas and thus obtain emission of nuclear magnetic resonance signals,
acquire nuclear magnetic resonance signals in the k space in the form of N acquisitions each passing through the centre of the k space and with an acquisition time N.TR for each k space (where TR is the repetition time separating two acquisitions),
and, starting from nuclear magnetic resonance signals, reconstruct images, replacing at least one acquisition of a k space for each image by an acquisition of the next k space.

2. Process according to claim 1, characterised in that it comprises means of acquisition of nuclear magnetic resonance signals in the k space in the form of N line acquisitions each passing through the centre of the k space.

3. Process according to claim 1, characterised in that it comprises means of acquisition of nuclear magnetic resonance signals in the k space, in the form of N acquisitions of spiral curves each passing through the centre of the k space.

4. Process according to claim 1, characterised in that it consists of building up images by replacing at least the first acquisition of a k space for each image by the first acquisition of a next k space.

5. Process according to claim 1, characterised in that it is designed to cyclically vary the normal direction to the imagery plane in which magnetic field gradients are applied, to acquire the k space.

6. Process according to claim 1, characterised in that it is designed to vary the normal direction to the imagery plane in which magnetic field gradients used to acquire the k space are applied, for each N acquisition.

7. Installation for the production of magnetic resonance images of a body for which an image is required, characterized in that it comprises:
means of injecting a blood tracer containing at least one hyperpolarized rare gas into the blood circulatory system of at least one body for which an image is required,
and a device for the production of magnetic resonance images comprising:
means of production of a stationary magnetic field,
a high frequency coil system in order to produce radio frequency pulses,
a gradient coil system in order to produce magnetic fields with gradients,
means of acquisition of nuclear magnetic resonance signals in the k space in the form of N acquisitions each passing through the centre of the k space and with an acquisition time N.TR for each k space (where TR is the repetition time separating two acquisitions),
and means of reconstructing images starting from nuclear magnetic resonance signals, by replacing at least one acquisition of a k space for each image by an acquisition of a next k space.

8. Installation according to claim 7, characterised in that the image production device comprises means of acquisition of nuclear magnetic resonance signals in the k space in the form of N line acquisitions each passing through the centre of the k space.

9. Installation according to claim 7, characterised in that the image production device comprises means of acquisition of nuclear magnetic resonance signals in the k space in the form of N acquisitions of spiral curves each passing through the centre of the k space.

10. Installation according to claim 7, characterised in that the magnetic resonance image production device comprises means of reconstructing images starting from nuclear magnetic resonance signals, by replacing at least one acquisition of a k space for each image by an acquisition of the next k space.

11. Installation according to claim 7, characterised in that the magnetic resonance image production device comprises means of cyclically varying the normal direction to the imagery plane in which the magnetic field gradients used to acquire the k space are applied.

12. Installation according to claim 7, characterised in that the magnetic resonance image production device, comprises means of varying the normal direction to the imagery plane in which the magnetic field gradients used to acquire the k space are applied, for each N acquisition.

13. Process according to claim 1, applied for production of magnetic resonance images of the heart.

14. Process according to claim 1, applied for production of magnetic resonance images of coronaries or infusion of the myocardium.
